(19) United States
(12) Patent Application Publication
(10) Pub. No.: US 2018/0080923 A1
(43) Pub. Date: Mar. 22, 2018

(54) Toilet Bowl Optical Engine

(71) Applicants: David R. Hall, Provo, UT (US); Dan Allen, Springville, UT (US); Joe Fox, Spanish Fork, UT (US)

(72) Inventors: David R. Hall, Provo, UT (US); Dan Allen, Springville, UT (US); Joe Fox, Spanish Fork, UT (US)

(21) Appl. No.: 15/269,207

(22) Filed: Sep. 19, 2016

(51) Int. Cl. G01N 33/493; E03D 11/02; G01N 21/55; G01N 21/59; A61B 10/00

(52) U.S. Cl. CPC G01N 33/493; E03D 11/02; G01N 21/55; G01N 2201/0683; A61B 10/007; G01N 2201/0231; G01N 21/59

(57) ABSTRACT

An optical engine directly attached to a surface of a toilet bowl is disclosed. An optical element of the optical engine traps urine for analysis. A cleaning jet may be used to clean and dry the optical element after urine is analyzed. A heater may be used to preheat a urine capture area before receiving urine and control urine temperature while testing the urine. User feedback associated with urinalysis results may be visually given to a toilet user by one or more light sources of the optical engine.

### BACKGROUND

### Field of the Invention

The present invention relates to the function and utility of in-toilet urine capture and measurement.

### Background of the Invention

Urinalysis is a cheap, fast and simple screening tool for many health conditions. The urinalysis machine is large and expensive and inconvenient for users (pee in a cup). A method for analyzing urine, providing a variety of health-relevant measurements in a toilet is needed.

### SUMMARY

This invention has been developed in response to the present state of the art and, in particular, in response to the problems and needs in the art that have not yet been fully solved by currently available systems and methods. Accordingly, a toilet bowl with optical engine has been developed. Features and advantages of different embodiments of the invention will become more fully apparent from the following description and appended claims, or may be learned by practice of the invention as set forth hereinafter.

According to the invention, an optical engine is directly attached to a surface of a toilet bowl. The optical engine comprising a first light source, and a first detector. A portion of the urine is captured and analyzed on an optical element of the optical engine. User feedback of the analyzed urine is given visually on or near the optical element when the analysis is complete. An optical element of the optical engine may form at least part of a slit, indentation, trench, pattern, divot, concavity, prism, lens array, or diffraction grating where the urine is analyzed. One or more temperature sensors may contact the optical element or the urine. The temperature sensors may be used to detect the urine entering the toilet and to control a temperature of the urine. A cleaning jet may be used to clean and dry the optical element. The cleaning jet may spray water, air, or a combination thereof. The light engine may comprise one or more polarizers, patterned polarizers, lenses, micro-lens arrays, patterned micro-lens arrays, diffraction gratings, nano-structured optical elements, optical retarders, nano-structured patterned lenses, light collimators, or light detectors. An optical element used to assist in analyzing the urine may comprise one or more polarizers, patterned polarizers, lenses, micro-lens arrays, patterned micro-lens arrays, diffraction gratings, nano-structured optical elements, cover glass, beam splitting cubes, optical interfaces, mirrors, optical retarders, nano-structured patterned lenses, fiber optic lines, fiber optic interfaces, light collimators, or light detectors. The optical element may comprise a hydrophobic surface coating or a hydrophilic surface coating. The toilet may comprise a cleaning jet which cleans the recess. The cleaning jet may be located above the recess. The cleaning jet may spray water, air, cleaning solution, or a combination thereof to clean and dry the recess. A cleaning solution may be used in combination with water, air, or a combination of water and air to clean the recess. The optical engine may comprise a first beam splitter. The optical engine may comprise a second beam splitter. The first beam splitter and the second beam splitter may be polarization beam splitters. The first beam splitter and the second beam splitter may be coaxially located on an optical axis. The optical engine may comprise a second light source. The optical engine may further comprise a second detector. The optical engine may further comprise a second light source, a second detector, and a first beam splitter. The optical engine may use both reflection and transmission modes to analyze the urine. A controller may be operably connected to the optical engine. The controller may comprise a wireless or wired transceiver. The toilet may comprise a heater in thermal communication with the optical element. The heater may be resistive or inductive. The optical engine may be glued to a surface of the bowl. The surface may be an inner bowl surface or an outer bowl surface. Optical feedback may be provided to the toilet user of an out-of-range urine measurement by visually modulating or illuminating a urine capture area. The illumination may indicate to the user that the results are out of a normal range and they need to look at the full urinalysis results. The illumination may indicate to the user that the results are normal and they don't need to look at the full urinalysis results. A red light may indicate that at least one of the urinalysis results are out of range and a green light may indicate that all of the results are in a normal range. A portion of the received urine may be captured and analyzed on an optical element of the optical engine. An optical engine may include refractometers, spectrometers, glucose polarimeters, laser scatterometers, turbidity detectors, microscopes, or a combination thereof. An optical element of the optical engine may form at least part of a slit, indentation, trench, pattern, divot, concavity, prism, lens array, or diffraction grating. An optical engine may include one or more amplitude modulated light source. An optical engine may share one or more light sources with multiple detectors. An optical element of the optical engine may include a trap region that traps urine via surface tension. The trap region may comprise a longitudinal dimension in a longitudinal direction and a transverse dimension in an orthogonal transverse direction, the longitudinal dimension being at least twice the transverse dimension. Two or more detectors within the optical engine may share an amplitude modulated light source. The recess may form a slit, groove, indentation, trench, pattern, divot, concavity, prism, lens, lens array, or diffraction grating. The recess may comprise a hydrophobic surface coating or a hydrophilic surface coating. The toilet may comprise a cleaning jet which cleans the recess. The cleaning jet may spray water, air, or a combination thereof. The toilet may comprise a cleaning jet which cleans the recess. The cleaning jet may be located above the recess. The cleaning jet may spray water, air, cleaning solution, or a combination thereof to clean and dry the recess. A cleaning solution may be used in combination with water, air, or a combination of water and air to clean the recess. The recess may comprise a temperature sensor. The optical engine may comprise a temperature sensor. The temperature sensors may be used to detect the urine entering the recess and to control a temperature of the recess. The optical engine may use a shared optical path between multiple detectors. The optical engine may comprise one or more beam splitter. The beam splitters may be polarization beam splitters or non-polarization beam splitters or a combination thereof. One or more controllers may be operably connected to the optical engine. The one or more controllers may each comprise a wireless or wired transceiver. The optical engine or toilet may comprise a heater. The toilet may comprise a heater in thermal communication with the optical element. The heater may be resistive or inductive. The optical engine may be glued to a surface of the bowl.

In an example optical engine, a collimated polarized light source, such as laser light collimated from a single mode fiber passes through the urine trapped in a recess. The beam may be divided by a non-polarizing beam-splitters and sent to a microscope objective to relay the beam to a camera for heterodyne near field light scattering measurements, useful for determining the particle size distribution in the urine. Hemoglobin, leukocytes and oxalate crystals are quite different in size and arise from different health conditions (kidney damage, urinary tract infection and kidney stone problems, respectively, for an example. The light scattering instrument path is bent downward by the beam splitter to create more space for the potentially bulky objective lens. Part of the laser beam may pass to a polarizing beam splitter allowing a differential measurement of the polarization state of the laser, which will depend on the glucose concentration, primarily. A differential measurement is particularly sensitive to small changes in polarization state and is suitable for shorter path length measurements than typical polarimeters which use 10 cm path length. A refractometer may be constructed from an LED, an optional aperture, an optical coupling element such as a prism and a detector with spatial resolution such as a segemented photodiode, position sensitive photodiode or camera. A range of incident angles may strike the urine from the bottom. Some rays totally internally reflect and create a bright portion on a detector. Other rays partially transmit and provide a lower level of illumination. A polarizer can optionally be included for better resolution. A refractometer may be oriented such the light path is in the plane of the long direction of the slit, rather than a short direction with advantages for optical alignment. A camera may also be used to detect scattered light from a laser, for measuring turbidity. A multi-wavelength light source and wavelength-sensitive detection can be included for spectroscopic or color measurements, as described hereinafter. A microscope may be used to accomplish microscopic analysis of the urine. Optical instruments within the optical engine may share a light source with homodyne near field scattering. Detectors may be masked photodiodes or (CCD, LCOS, CMOS) cameras when discrimination against a split out light path is desired. For robustness the entire optical engine assembly may be cemented together.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings, in which:

FIG. 1 shows an optical engine assembly attached to a toilet bowl surface;

FIG. 2 shows an optical engine assembly attached to a toilet bowl surface;

FIG. 3 shows an optical engine assembly attached to a toilet bowl surface;

FIG. 4 shows a urine recess trap according to an embodiment of the invention;

FIG. 5 shows an optical engine assembly attached to a toilet bowl surface according to an embodiment of the invention;

FIG. 6 shows an optical engine assembly attached to a toilet bowl surface;

FIG. 7 shows a toilet bowl with a urine recess trap according to an embodiment of the invention;

FIG. 8 shows an optical engine assembly attached to a toilet bowl surface;

FIG. 9 shows an optical engine assembly attached to a toilet bowl surface; and

FIG. 10 shows a flow diagram of detecting, measuring, and providing feedback to a user in accordance with an embodiment of the invention.

### DETAILED DESCRIPTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

A detailed description of the claimed invention is provided below by example, with reference to embodiments in the appended figures. Those of skill in the art will recognize that the components of the invention as described by example in the figures below could be arranged and designed in a wide variety of different configurations. Thus, the detailed description of the embodiments in the figures is merely representative of embodiments of the invention, and is not intended to limit the scope of the invention as claimed.

In some instances, features represented by numerical values, such as dimensions, mass, quantities, and other properties that can be represented numerically, are stated as approximations. Unless otherwise stated, an approximate value means “correct to within 50% of the stated value.” Thus, a length of approximately 1 inch should be read “1 inch+/−0.5 inch.”

Aspects of the present invention are described herein with reference to flowchart illustrations and/or block diagrams of methods, apparatus (systems), and computer program products according to embodiments of the invention. Those of skill in the art will understand that each block of the flowchart illustrations and/or block diagrams, and combinations of blocks in the flowchart illustrations and/or block diagrams, may be implemented by computer readable program instructions. Additionally, those of skill in the art will recognize that the system blocks and method flowcharts, though depicted in a certain order, may be organized in a different order and/or configuration without departing from the substance of the claimed invention.

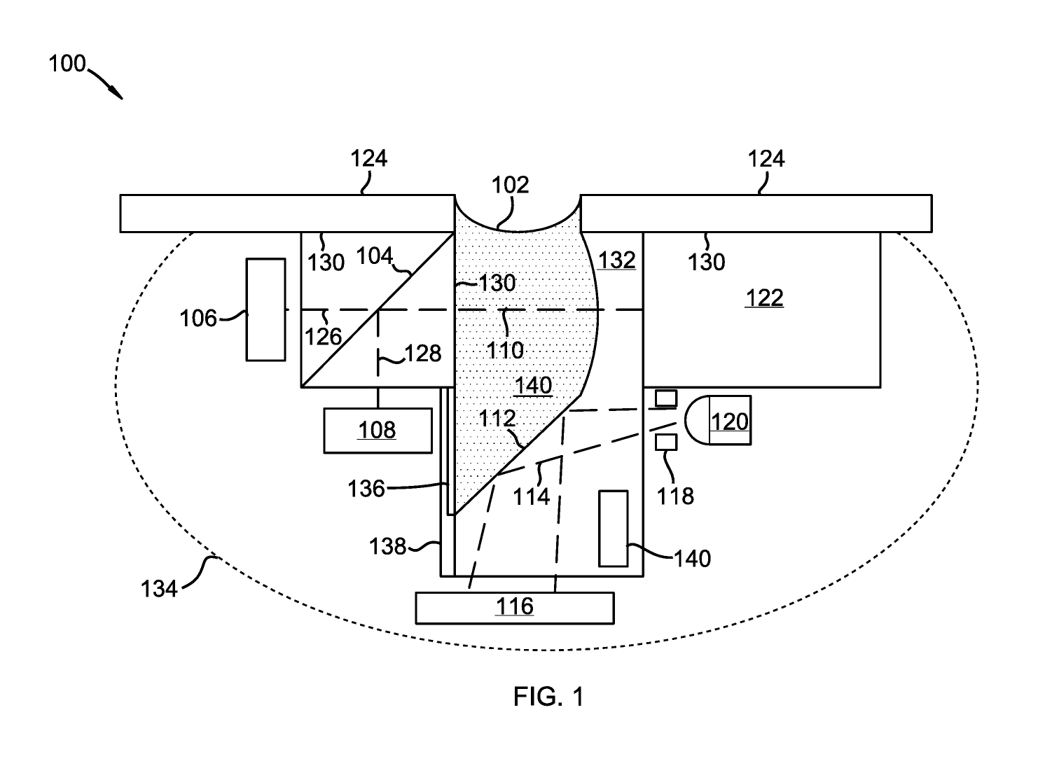

FIG. 1 shows an optical engine 134 including urine 102 in a recess 140, refractometer detector 116, dynamic light scattering detector 116, turbidity sensor 116, light scattering particle sizer 116, color sensor 116, glucose polarimeter detectors 106/108, and beam splitter 104. The recess 140 that houses urine 102 may be formed by a slit in an optically transparent material such as glass, quartz, or plastics. Light source 122, refractometer detector 116, optical element 132, dynamic light scattering detector 116, turbidity sensor 116, light scattering particle sizer 116, color sensor 116, glucose polarimeter detectors 106/108, temperature sensors 136/138, and beam splitter 104 may entirely or partially form the recess. Optical element 132 may form at least part of a slit, indentation, trench, pattern, divot, concavity, prism, lens array, or diffraction grating. An optical element 132 may be used to assist in analyzing the urine and may comprise one or more polarizers, patterned polarizers, lenses, micro-lens arrays, patterned micro-lens arrays, diffraction gratings, nano-structured optical elements, cover glass, beam splitting cubes, optical interfaces, mirrors, optical retarders, nano-structured patterned lenses, fiber optic lines, fiber optic interfaces, light collimators, or light detectors. Light sources 122 and 120 may be a collimated laser, multiple collimated lasers of different frequency, one or more laser diodes of different frequencies, or a white light source which is polarized and collimated. Detectors 106, 108, 116 may be pixelated sensor arrays such as LCOS (liquid crystal on silicon), CMOS (complementary metal-oxide-semiconductor), CCD (capacitive coupled display), or arrays of photo-diodes. Beam splitter 104 may serve as a polarization beam splitter and separate polarization states which are orthogonal to each other, reflecting the different polarization states respectively to each detector 106 and 108. Beam splitter 104 may be a cube beam splitter employing thin films or a wire-grid polarizer.

Toilet bowl urine measurements may be taken when a toilet user urinates in a toilet and urine contacts urine trapping area 140. The urine may directly hit trapping area 140 as released by a toilet user or the urine may travel along an inside surface 124 of a toilet and become trapped in area 140. One or more temperature sensors 136 and 138 located in trapping area 140 may detect urine and trigger measurement devices 106, 108, 116, 120 and 122 to measure urine trapped in area 140. A heater 140 may be positioned within or near trapped area 140. A toilet controller may preheat area 140 and keep area 140 at a fixed temperature while performing urine testing. When measurements are complete, flush water released from the toilet may be used to clean trapped urine in area 140. A toilet controller may contain programming to wait for measurement devices 106, 108, 116, 120, and 122 to complete urine measurements before allowing the toilet to flush. For example, a user may push the flush button and a toilet controller may delay the flush until it receives acknowledgement that the urine measurements are complete. Optical feedback may be provided to the toilet user of an out-of-range urine measurement by visually modulating a urine capture area indicating to the user that they need to look at the urinalysis results. This may be accomplished by turning on a colored led or laser light from sources 122 or 120. A red light may mean that at least one of the urinalysis results are out of range and a green light may indicate that all of the results are in a normal range.

Measurement system 100 may include one or more controllers, processors, light sources, lenses, diffraction optics, collimating optics, power sources, and light detectors. Power sources may be battery power, generator power, or a wired power connection. Each controller may contain wireless and wired transceivers for communicating data to remote computers, user devices, and remote databases. Data may be communicated over the Internet or over local networks and devices. Urine trapping recess area 140 may be formed by a slit, groove, recess, indentation, trench, pattern, divot, concavity, prism, lens, lens array, and/or diffraction grating. Optical engine 134 may be formed into a single rigid optical engine module and may be glued or fastened to an interior and/or exterior surface 130 of a toilet bowl 124. Light sources 122 and/or 120 may illuminate red or green lighting at the end of a urine testing indicating an out of tolerance result or indicating a normal result respectively, allowing a user to obtain a visual indication of a normal or abnormal urine test.

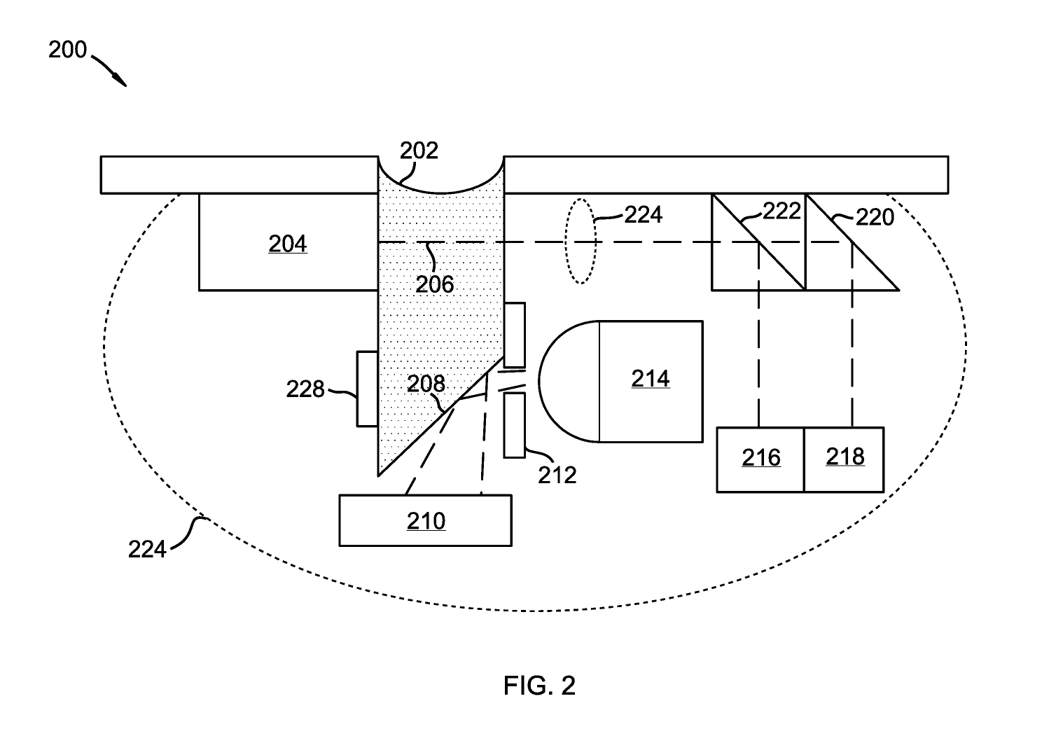

FIG. 2 shows an optical engine arrangement including urine 202 in a recess, with refractometer detector 210, dynamic light scattering detector 210, turbidity sensor 210, light scattering particle sizer 210, color sensor 210, glucose polarimeter detectors 216/218, and beam splitters 222 and 220. The recess that houses urine 202 may be formed by a slit in an optically transparent material such as glass, quartz, or plastics. Light source 204, refractometer detector 210, dynamic light scattering detector 210, turbidity sensor 210, light scattering particle sizer 210, color sensor 210, glucose polarimeter detectors 216/218, and beam splitters 222 and 220 may entirely or partially form the recess. Light sources 204 and 214 may be a collimated laser, multiple collimated lasers of different frequency, one or more laser diodes, or a white light source which is polarized and collimated. Surface 208 may be formed as part of a prism and used to assist in reflection of light from light source 214 to detector 210. Detectors 210, 218, 216 may be pixelated sensor arrays such as LCOS (liquid crystal on silicon), CMOS (complementary metal-oxide-semiconductor), CCD (capacitive coupled display), or arrays of photo-diodes. Beam splitter 222 may serve as a polarization beam splitter and separate polarization states which are orthogonal to each other, reflecting the different polarization states respectively to each detector 216 and 218. Beam splitter 222 may be a cube beam splitter employing thin films or a wire-grid polarizer. Beam splitter 220 may use a silvered mirror and/or thin film interfaces to reflect light.

Measurement system 200 may contain one or more controllers, processors, light sources, lenses, diffraction optics, collimating optics, power sources, and light detectors. Power sources may be battery power, generator power, or a wired power connection. Each controller may contain wireless and wired transceivers for communicating data to remote computers, user devices, and remote databases. Data may be communicated over the Internet or over local networks and devices. The urine 202 trapping recess area may be formed by a slit, groove, recess, indentation, trench, pattern, divot, concavity, prism, lens, lens array, and/or diffraction grating. Optical engine 224 may be formed into a single rigid optical engine module and may be glued or fastened to an interior and/or exterior surface of a toilet bowl. Light sources 204 and/or 214 may illuminate red or green lighting at the end of a urine testing indicating an out of tolerance result or indicating a normal result respectively, allowing a user to obtain a visual indication of a normal or abnormal urine test.

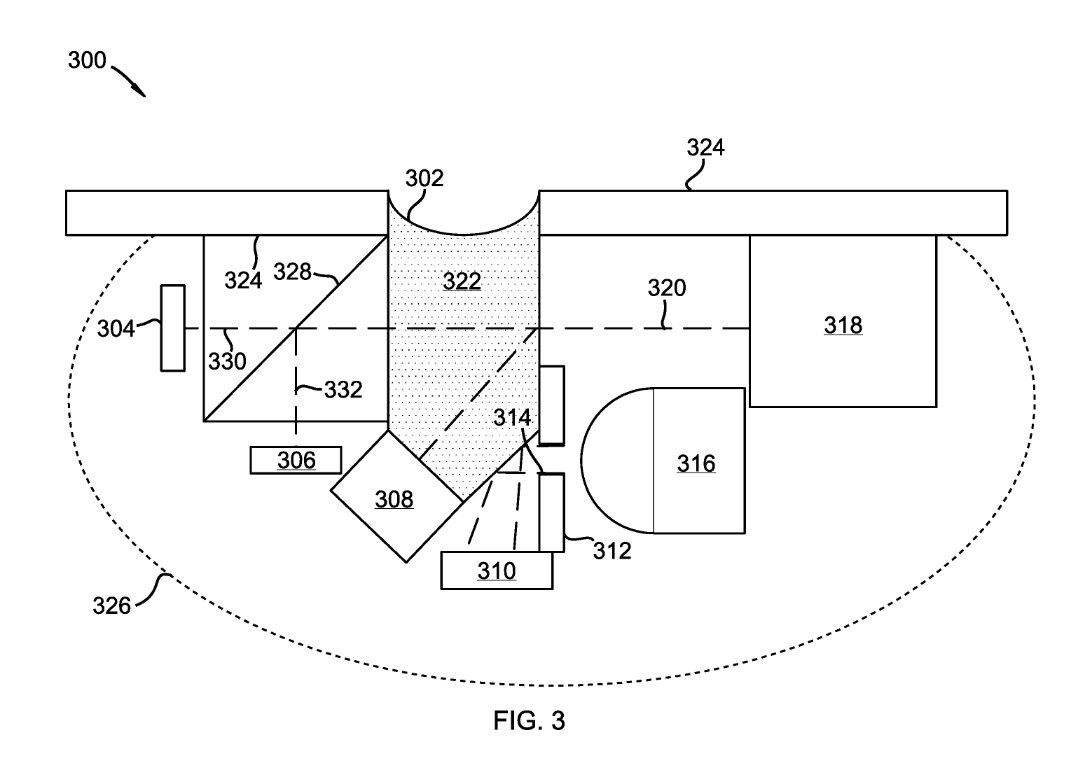

FIG. 3 shows an optical engine arrangement including urine 302 in a recess 322, with refractometer detector 310, dynamic light scattering detector 310, turbidity sensor 310, light scattering particle sizer 310, color sensor 310, glucose polarimeter detectors 304/306, and beam splitter 328. The recess 322 that houses urine 302 may be formed by a slit in an optically transparent material such as glass, quartz, or plastics. Light source 318, refractometer detector 310, dynamic light scattering detector 310, turbidity sensor 310, light scattering particle sizer 310, color sensor 310, glucose polarimeter detectors 304/306, and beam splitter 328 may entirely or partially form the recess. Light sources 318, 316 and 308 may be a collimated laser, multiple collimated lasers of different frequency, one or more laser diodes of different frequencies, or a white light source which is polarized and collimated. Detectors 310, 304, 306 may be pixelated sensor arrays such as LCOS (liquid crystal on silicon), CMOS (complementary metal-oxide-semiconductor), CCD (capacitive coupled display), or arrays of photo-diodes. Beam splitter 328 may serve as a polarization beam splitter and separate polarization states which are orthogonal to each other, reflecting the different polarization states respectively to each detector 304 and 306. Beam splitter 328 may be a cube beam splitter employing thin films or a wire-grid polarizer.

Measurement system 300 may contain one or more controllers, processors, light sources, lenses, diffraction optics, collimating optics, power sources, and light detectors. Power sources may be battery power, generator power, or a wired power connection. Each controller may contain wireless and wired transceivers for communicating data to remote computers, user devices, and remote databases. Data may be communicated over the Internet or over local networks and devices. Urine trapping recess area 322 may be formed by a slit, groove, recess, indentation, trench, pattern, divot, concavity, prism, lens, lens array, and/or diffraction grating.

FIG. 3 shows an optical arrangement according to an embodiment of the invention. Light source 304 may transmit polarized light 306 through urine 302. Beam splitter 326 is a non-polarization beam splitter such as a 50% silvered mirror or a thin film filter. When polarized light 306 is incident on beam splitter 326, 50% is transmitted to polarization beam splitter 324 to detectors 322 and 320 and 50% is reflected through lens 318 to detector 316. Detectors 320 and 322 may be used as part of a glucose polarimeter. Polarization rotation change as a result of light 306 passing through urine 302 can be detected by detectors 320 and 322. Detector 316 is used to detect particle size. Detectors 320, 322, 316 may be pixelated sensor arrays such as LCOS (liquid crystal on silicon), CMOS (complementary metal-oxide-semiconductor), CCD (capacitive coupled display), or arrays of photo-diodes.

A refractometer comprising a light source 308, an aperture prism 310 and detector 312 may be positioned so the light transmitted from light source 308 is transmitted perpendicularly (the long direction of urine slot) to the light transmitted by light source 304. FIG. 3 shows refractometer 308, 310, and 312 in the same plane as 304, but the refractometer system may be coming out of the page in the “z” direction. Transmitting and detecting light in the long direction saves space and also provides more cross-sectional area for refractometer and turbidity measurements. Light sources 318, 308, and/or 316 may illuminate red or green lighting at the end of a urine testing indicating an out of tolerance result or indicating a normal result respectively, allowing a user to obtain a visual indication of a normal or abnormal urine test.

Measurement instruments 300 may contain one or more controllers, processors, light sources, lenses, diffraction optics, collimating optics, power sources, and light detectors. Power sources may be battery power, generator power, or a wired power connection. Each controller may contain wireless and wired transceivers for communicating data to remote computers, user devices, and remote databases. Data may be communicated over the Internet or over local networks and devices. The urine 302 trapping recess area may be formed by a slit, groove, recess, indentation, trench, pattern, divot, concavity, prism, lens, lens array, and/or diffraction grating.

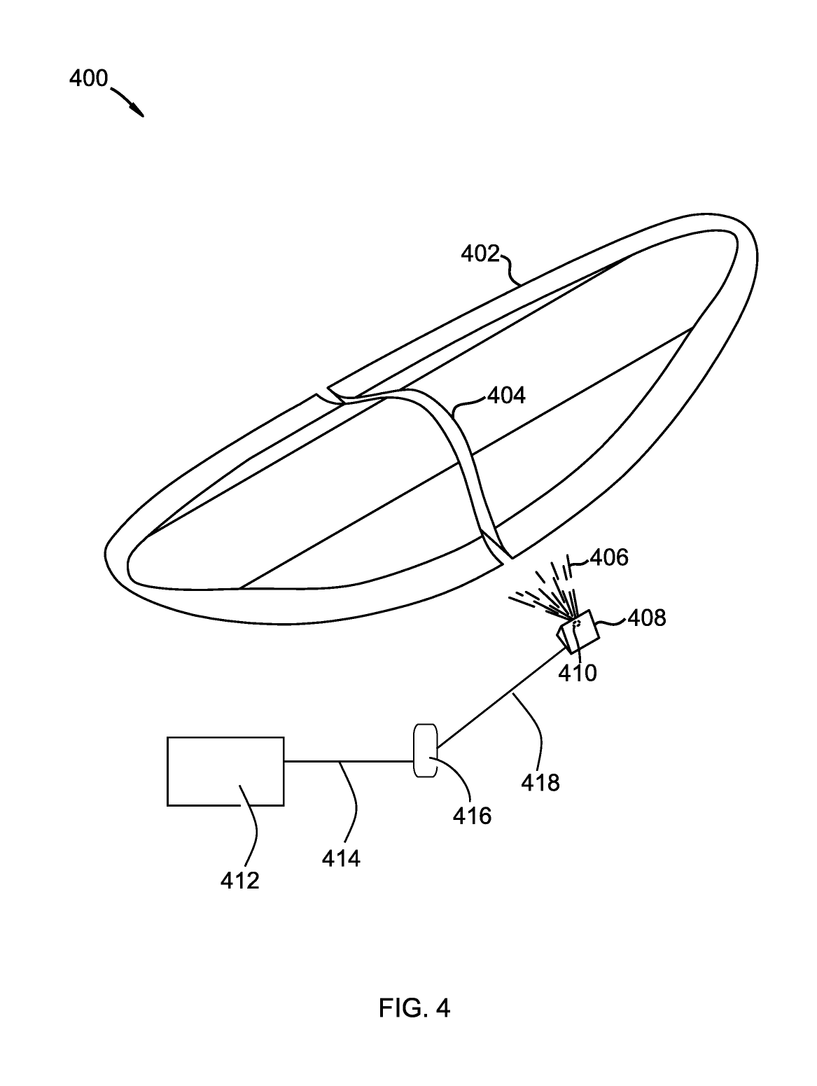

FIG. 4 shows a urine trapping device 400 with a cleaning jet 408, a toilet controller 412, one or more solenoids 416, and bowl connection surface 402. The slot 404 may comprise a trap region 404 that traps urine via surface tension. The trap region 404 may comprise a longitudinal dimension in a longitudinal direction and a transverse dimension in an orthogonal transverse direction, the longitudinal dimension being at least twice the transverse dimension. The cleaning jet 408 may be operably connected 414/418 to a toilet controller 412 and a solenoid valve 416. The controller 412 may be programmed to actuate one or more solenoids 416 allowing air, water, or a combination of air and water to spray a j et stream 406 into the urine trap area effectively cleaning the urine out of the trap area and leaving the trap area dry. The toilet may comprise a cleaning jet 408 which cleans the recess 404. The cleaning jet 408 may be located above the recess. The cleaning jet may spray water, air, cleaning solution, or a combination thereof to clean and dry the recess. A cleaning solution may be used in combination with water, air, or a combination of water and air to clean the recess. The controller 412 may alternate cleaning and drying of the trap area 404 for a predetermined number of iterations such as 5 or 10. Bowl connection surface 402 may be glued or fastened to an interior and/or exterior surface of the toilet bowl.

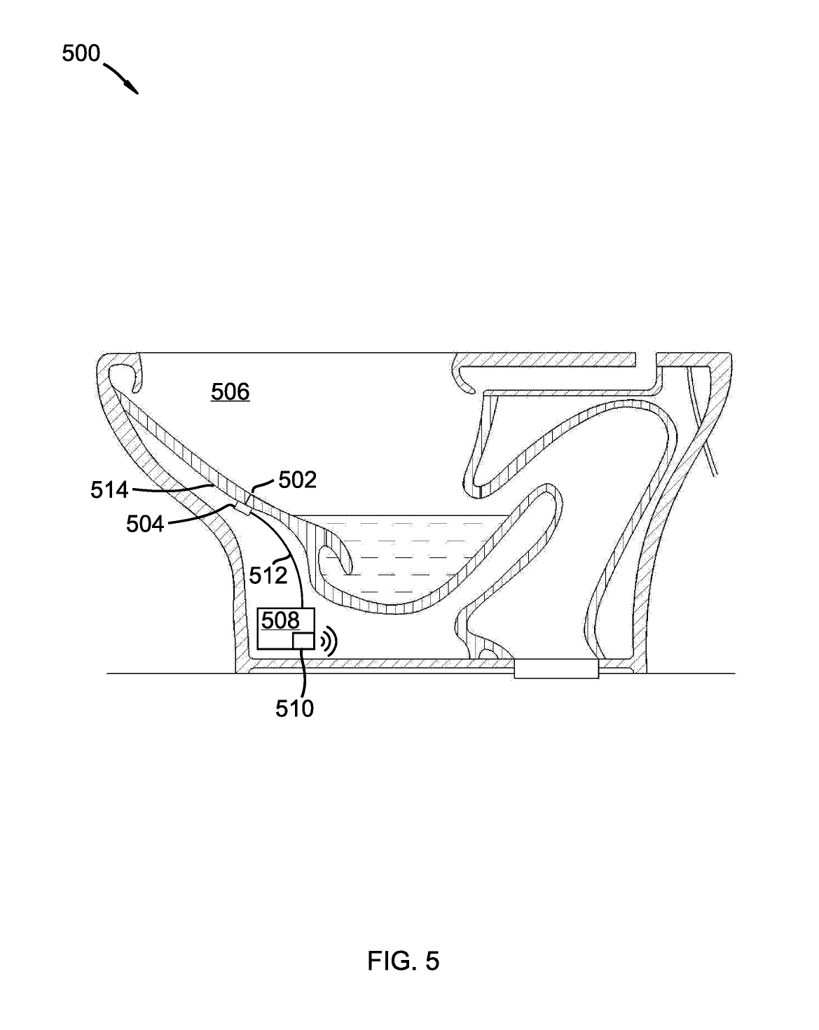

Referring to FIG. 5, a toilet bowl measurement system 500 with optical engine 504 is depicted with a urine trapping recess area 502 and communication lines 512. Optical engine 504 may comprise refractometers, spectrometers, glucose polarimeters, laser scatterometers, turbidity detectors, and/or microscopes. Measurement system 500 may contain one or more controllers 508, power sources, and wireless transceivers 510. The controller may control measurement functions of optical engine 504 in addition to toilet flushing functions. Power sources may include battery, generator, or a wired power connection. The controller 508 may contain wireless and wired transceivers 510 for communicating data to remote computers, user devices, and remote databases. Data may be communicated over the Internet or over local networks and devices. Urine trapping recess area 502 may be formed by a slit, groove, recess, indentation, trench, pattern, divot, concavity, prism, lens, lens array, and/or diffraction grating. Communication lines 512 may include electrical wires and data wires.

Toilet bowl urine measurements may be taken when a toilet user urinates in toilet bowl 506 and urine contacts urine trapping area 502. The urine may directly hit trapping area 502 as released by a toilet user or the urine may travel along an inside surface of toilet bowl and become trapped in area 502. A temperature sensor may be located in trapping area 502 and may detect urine and trigger optical engine or controller 508 to measure urine trapped in area 502. A heater may be positioned within or near trapped area 502. A toilet controller 508 may preheat area 502 and keep area 502 at a fixed temperature while performing urine testing. When measurements are complete, flush water released from toilet bowl 506 may be used to clean trapped urine in area 502. A toilet controller 508 may contain programming to wait for optical engine 504 to complete urine measurements before allowing the toilet to flush. For example, a user may push the flush button and a toilet controller 508 may delay the flush until it receives acknowledgement that the urine measurements are complete.

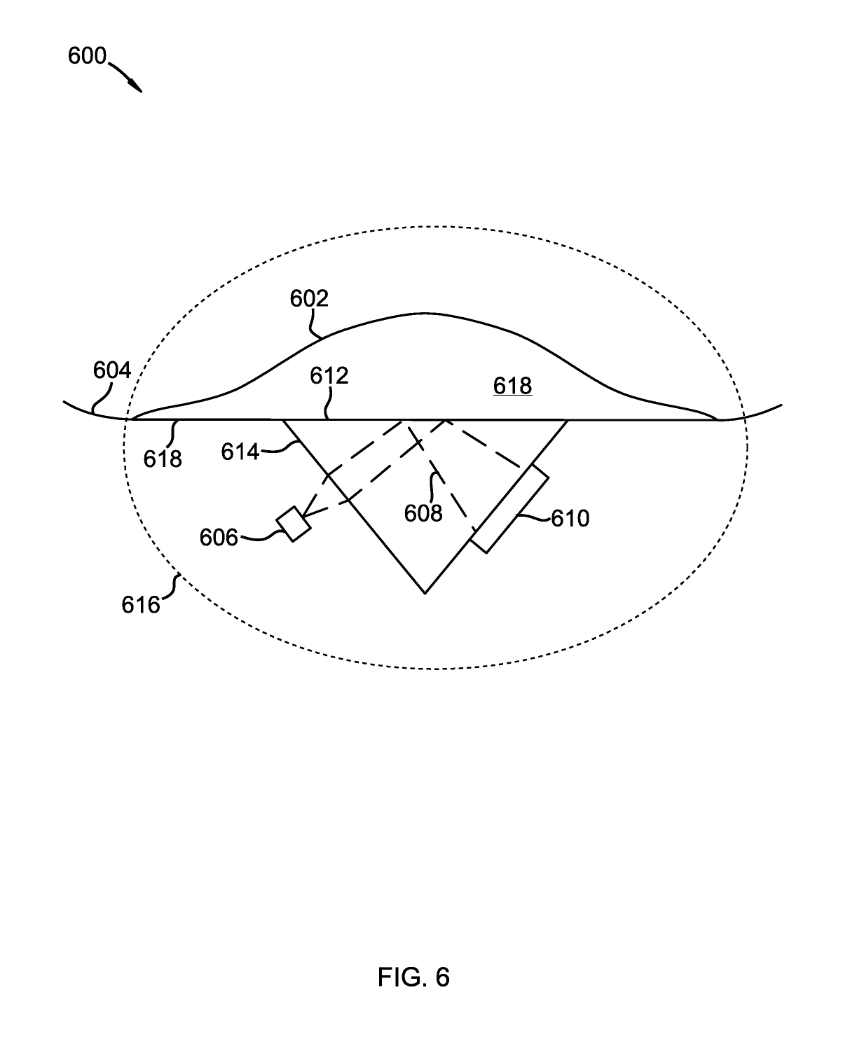

FIG. 6 shows an optical engine arrangement 616 including a recess 602, with refractometer detector 610, dynamic light scattering detectors 610, turbidity sensors 610, light scattering particle sizers 610, and color sensors 610. The recess that houses urine 602 may be formed by a slit in an optically transparent material such as glass, quartz, or plastics. Light sources 606, refractometer detector 610, dynamic light scattering detector 610, turbidity sensor 610, light scattering particle sizer 610, and color sensor 610 may entirely or partially form the recess. Light sources 606 may be a collimated laser, multiple collimated lasers of different frequency, one or more laser diodes of different frequencies, or a white light source which is polarized and collimated. Multi-angle, multiple wavelength measurements are enabled as a result of detectors 610 and multiple wavelength light source 606. Detectors 610 be pixelated sensor arrays such as LCOS (liquid crystal on silicon), CMOS (complementary metal-oxide-semiconductor), CCD (capacitive coupled display), or arrays of photo-diodes. Light source 606 may illuminate red or green lighting at the end of a urine testing indicating an out of tolerance result or indicating a normal result respectively, allowing a user to obtain a visual indication of a normal or abnormal urine test.

Measurement system 600 may contain one or more controllers, processors, light sources, lenses, diffraction optics, collimating optics, power sources, and light detectors. Power sources may be battery power, generator power, or a wired power connection. Each controller may contain wireless and wired transceivers for communicating data to remote computers, user devices, and remote databases. Data may be communicated over the Internet or over local networks and devices. The urine trapping recess area may be formed by a slit, groove, recess, indentation, trench, pattern, divot, concavity, prism, lens, lens array, and/or diffraction grating.

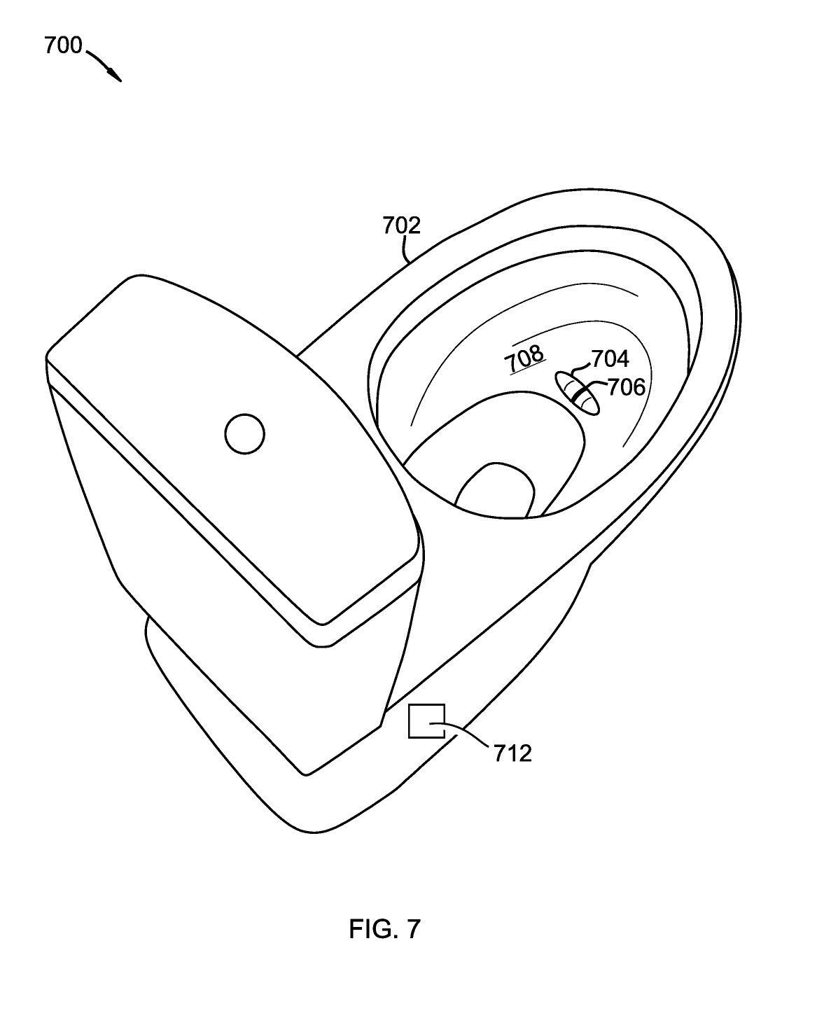

FIG. 7 shows a toilet 700 including a toilet bowl 702, an inner surface 708 of the toilet bowl, a recessed urine trapping device 704, an optical element slit 706, and a toilet controller 712. Recessed urine trapping device 704 is located above the bowl water level and still low enough to trap urine in a slot 706 in urine trapping device 704. The slit 706 may comprise a trap region that traps urine via surface tension. The slit 704 may comprise one or more one or more optical elements such as polarizers, patterned polarizers, lenses, micro-lens arrays, patterned micro-lens arrays, diffraction gratings, nano-structured optical elements, cover glass, beam splitting cubes, optical interfaces, mirrors, optical retarders, nano-structured patterned lenses, fiber optic lines, fiber optic interfaces, light collimators, or light detectors. The trap region may comprise a longitudinal dimension in a longitudinal direction and a transverse dimension in an orthogonal transverse direction, the longitudinal dimension being at least twice the transverse dimension. The cleaning jet may be operably connected to a toilet controller 712 and a solenoid valve. The controller 712 may be programmed to actuate one or more solenoids allowing air, water, or a combination of air and water to spray a jet stream into the urine trap area effectively cleaning the urine out of the trap area and leaving the trap area dry. The controller 712 may alternate cleaning and drying of the trap area for a predetermined number of iterations such as 5 or 10.

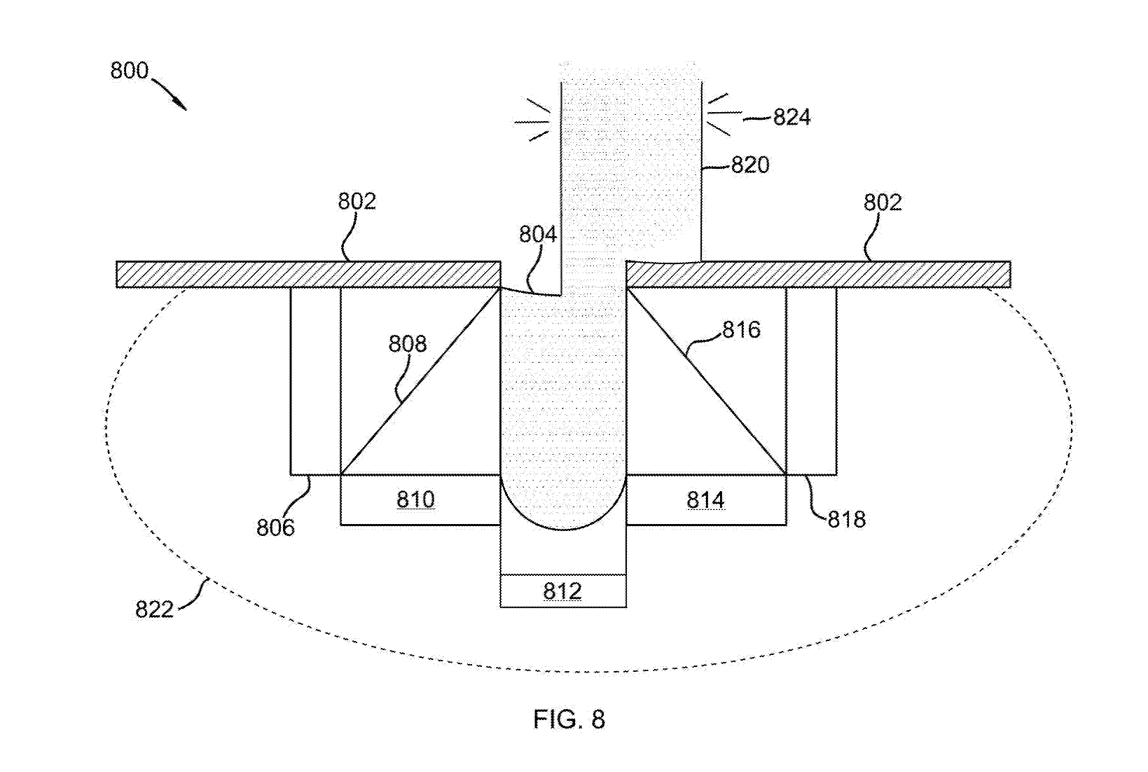

FIG. 8 shows an optical engine arrangement including urine 804 in a recess, with glucose polarimeter detectors 806/810/814/818, and beam splitters 808 and 816. The recess that houses urine 804 may be formed by a slit in an optically transparent material such as glass, quartz, or plastics. Light source 812, glucose polarimeter detectors 806/810/814/818, and beam splitters 808 and 816 may entirely or partially form the recess. Light source 812 may be a collimated laser, multiple collimated lasers of different frequency, one or more laser diodes, or a white light source which is polarized and collimated. Detectors 806/810/814/818 may be pixelated sensor arrays such as LCOS (liquid crystal on silicon), CMOS (complementary metal-oxide-semiconductor), CCD (capacitive coupled display), or arrays of photo-diodes. Beam splitters 808/816 may serve as a polarization beam splitter and separate polarization states which are orthogonal to each other, reflecting the different polarization states respectively to each detectors 806/810/814/818. Beam splitters 808 and 816 may be cube beam splitter employing thin films or wire-grid polarizers. Because light source 812 is directed toward urine stream 820, when urine stream 820 directly contact urine 804 within the recess, the urine stream 820 may light up illuminating 824 the urine stream 820 in an upward direction toward the person urinating indicating that the urine hit the correct location in the toilet bowl. A temperature sensor may detect urine entering the slot and turn on light source 812. Optical engine 822 may be formed into a single rigid optical engine module and be glued or fastened to a surface of a toilet bowl 802. Light source 812 may illuminate red or green lighting at the end of a urine testing indicating an out of tolerance result or indicating a normal result respectively, allowing a user to obtain a visual indication of a normal or abnormal urine test.

Measurement system 800 may contain one or more controllers, processors, light sources, lenses, diffraction optics, collimating optics, power sources, and light detectors. Power sources may be battery power, generator power, or a wired power connection. Each controller may contain wireless and wired transceivers for communicating data to remote computers, user devices, and remote databases. Data may be communicated over the Internet or over local networks and devices. The urine 804 trapping recess area may be formed by a slit, groove, recess, indentation, trench, pattern, divot, concavity, prism, lens, lens array, and/or diffraction grating.

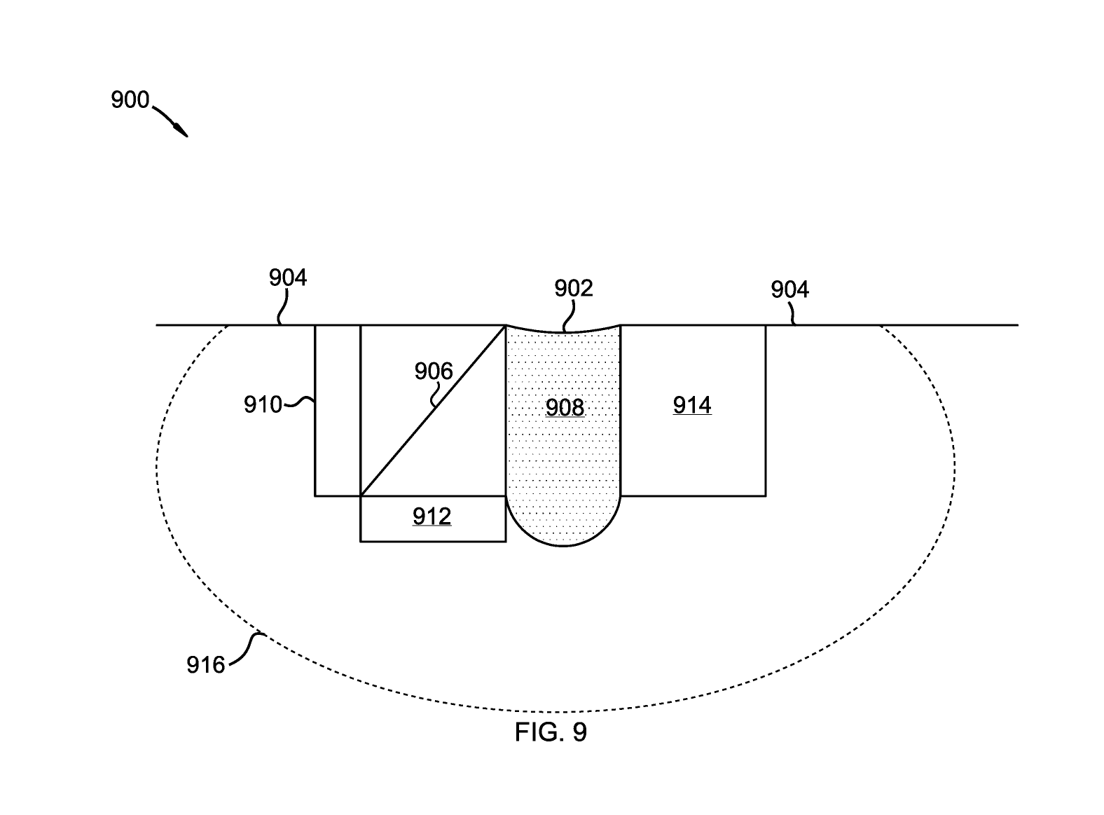

FIG. 9 shows an optical engine arrangement including urine 902 in a recess 908, with glucose polarimeter detectors 910/912, and beam splitter 906. The recess 908 that houses urine 902 may be formed by a slit in an optically transparent material such as glass, quartz, or plastics. Light source 914, glucose polarimeter detectors 910/912, and beam splitter 906 may entirely or partially form the recess. Light source 914 may be a collimated laser, multiple collimated lasers of different frequency, one or more laser diodes, or a white light source which is polarized and collimated. Detectors 910/912 may be pixelated sensor arrays such as LCOS (liquid crystal on silicon), CMOS (complementary metal-oxide-semiconductor), CCD (capacitive coupled display), or arrays of photo-diodes. Beam splitter 906 may serve as a polarization beam splitter and separate polarization states which are orthogonal to each other, reflecting the different polarization states respectively to each detectors 910/912. Beam splitter 906 may be cube beam splitter employing thin films or wire-grid polarizers. A temperature sensor may detect urine entering the slot and turn on light source 914. Optical engine 916 may be formed into a single rigid optical engine module and be glued or fastened to a surface of a toilet bowl 904. Light source 914 may illuminate red or green lighting at the end of a urine testing indicating an out of tolerance result or indicating a normal result respectively, allowing a user to obtain a visual indication of a normal or abnormal urine test.

Measurement system 900 may contain one or more controllers, processors, light sources, lenses, diffraction optics, collimating optics, power sources, and light detectors. Power sources may be battery power, generator power, or a wired power connection. Each controller may contain wireless and wired transceivers for communicating data to remote computers, user devices, and remote databases. Data may be communicated over the Internet or over local networks and devices. The urine 902 trapping recess area 908 may be formed by a slit, groove, recess, indentation, trench, pattern, divot, concavity, prism, lens, lens array, and/or diffraction grating.

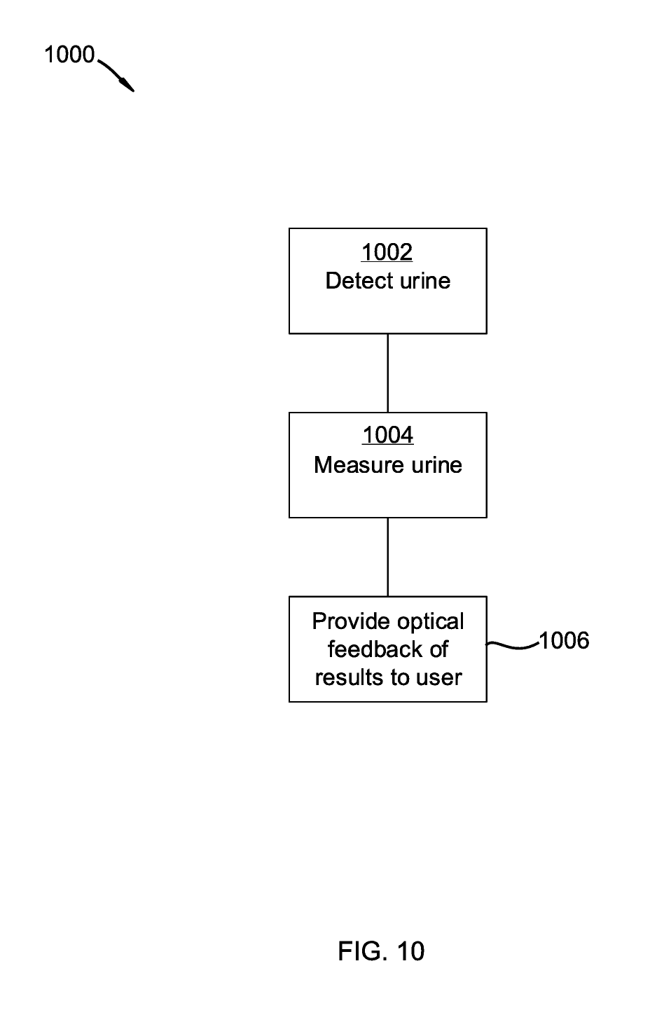

FIG. 10 shows a flow diagram of a urine detection, measurement, and feedback process. In step 1002, urine is detected. This detection may include using a temperature sensor located on or near a urine capture slot. Other forms of detection may include activation of a urine test by a user device such as an application program on a user smartphone, or motion detection by optical detectors within the optical engine, or light change detection by optical sensors within the optical engine. In step 1004, the urine is measured. The measurements may include refractometer measurements, spectrometer measurements, glucose polarimeter measurements, laser scatterometer measurements, turbidity detector measurements, microscope measurements, or a combination thereof. In step 1006, optical feedback may be provided to the toilet user. For instance, if the measurements detect an out-of-range measurement, a red light may continuously illuminate and/or blink in a urine capture area indicating to the user that they need to look at the urinalysis results. The results may be viewed online or on a user device operably connected to a toilet controller. The result may indicate that the user has a high number of leukocytes or any other urinalysis metric. If the results are in-range a green light may blink in a urine capture area, when testing is complete, indicating to the user that the results are in a normal range and no further action by the user is necessary.

The systems and methods disclosed herein may be embodied in other specific forms without departing from their spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A toilet comprising:
a bowl which receives urine;
an optical engine directly attached to a surface of the bowl, the optical engine comprising a first light source, and a first detector;
wherein a portion of the urine is captured and analyzed on an optical element of the optical engine; and
wherein user feedback of the analyzed urine is given visually on or near the optical element.

2. The toilet of claim 1, wherein the optical element forms at least part of a slit, indentation, trench, pattern, divot, concavity, prism, lens array, or diffraction grating.

3. The toilet of claim 1, further comprising a temperature sensor which contacts the optical element or the urine.

4. The toilet of claim 3, wherein the temperature sensor is used to detect the urine entering the toilet and to control a temperature of the urine.

5. The toilet of claim 1, further comprising a cleaning jet which cleans the optical element.

6. The toilet of claim 5, wherein the cleaning jet sprays water, air, or a combination thereof.

7. The toilet of claim 1, wherein the light engine comprises one or more polarizers, patterned polarizers, lenses, micro-lens arrays, patterned micro-lens arrays, diffraction gratings, nano-structured optical elements, optical retarders, nano-structured patterned lenses, light collimators, or light detectors.

8. The toilet of claim 1, wherein the optical element comprises a hydrophobic surface coating or a hydrophilic surface coating.

9. The toilet of claim 1, further comprising a first beam splitter.

10. The toilet of claim 9, further comprising a second beam splitter.

11. The toilet of claim 10, wherein the first beam splitter and the second beam splitter are polarization beam splitters.

12. The toilet of claim 11, wherein the first beam splitter and the second beam splitter are coaxially located on an optical axis.

13. The toilet of claim 1, further comprising a second light source.

14. The toilet of claim 1, further comprising a second detector.

15. The toilet of claim 1, further comprising a second light source, a second detector, and a first beam splitter.

16. The toilet of claim 15, wherein the optical engine uses both reflection and transmission modes to analyze the urine.

17. The toilet of claim 1, further comprising a controller operably connected to the optical engine.

18. The toilet of claim 17, wherein the controller comprises a wireless or wired transceiver.

19. The toilet of claim 1, further comprising a heater in thermal communication with the optical element.

20. The toilet of claim 1, wherein the optical engine is glued to the surface of the bowl.
